(19) United States
(12) Patent Application Publication
(10) Pub. No.: US 2014/0356416 A1
(43) Pub. Date: Dec. 4, 2014

(54) LIPOSOMAL DRUG ENCAPSULATION

(71) Applicant: The Regents of the University of California, Oakland, CA (US)

(72) Inventors: Santosh Kesari, San Diego, CA (US); Rajesh Mukthavaram, La Jolla, CA (US); Milan Makale, San Diego, CA (US); Wolf Wrasidlo, La Jolla, CA (US)

(21) Appl. No.: 14/299,662

(22) Filed: Jun. 9, 2014

Related U.S. Application Data
(63) Continuation of application No. PCT/US2012/068813, filed on Dec. 10, 2012.
(60) Provisional application No. 61/569,146, filed on Dec. 9, 2011.

(51) Int. Cl. A61K 31/553; A61K 9/127

(52) U.S. Cl. CPC A61K 31/553; A61K 9/127; A61K 9/1278
USPC 424/450; 514/211.08

(57) ABSTRACT

Provided herein are novel methods of making liposomally encapsulated drugs using reverse pH gradients and optimizing internal buffer compositions. Further provided herein are liposome compositions including an active pharmaceutical ingredient and uses thereof to treat a variety of diseases (e.g. cancer, inflammatory, neurological and cardiovascular diseases).

### CROSS-REFERENCES TO RELATED APPLICATIONS

This application claims the benefit of U.S. Provisional Application No. 61/569,146 filed Dec. 9, 2011, which is hereby incorporated in its entirety and for all purposes.

### STATEMENT AS TO RIGHTS TO INVENTIONS MADE UNDER FEDERALLY SPONSORED RESEARCH AND DEVELOPMENT

This invention was made with government support under CA023100 awarded by the National Institutes of Health. The Government has certain rights in the invention.

### BACKGROUND OF THE INVENTION

Liposome-based drug carriers can effectively enhance drug efficacy while reducing toxicity, and they have considerable potential as drug delivery platforms in cancer (Drummond, D. C. et al., Pharmacol Rev 1999, 51 (4), 691-743; Allen, T. M.; Cullis, P. R., Science 2004, 303 (5665), 1818-22). Examples include liposomal doxorubicin, and liposomal cytarabine, both FDA approved for cancer treatment (Gabizon, A. et al., J Control Release 1998, 53 (1-3), 275-9; Bomgaars, L. et al.; J Clin Oncol 2004, 22 (19), 3916-21). However, a key and pervasive obstacle is that many clinically promising drug classes are difficult to stably encapsulate within liposomes (Fritze, A. et al., Biochem Biophys Acta 2006, 1758 (10), 1633-40; Haran, G. et al., Biochim Biophys Acta 1993, 1151 (2), 201-15).

Staurosporine is a pan protein kinase inhibitor with potent anticancer activity in vitro, but clinical use of this compound is precluded by plasma protein binding with rapid clearance, and non-selective toxicity (Gurley L R et al., Staurosporine analysis and its pharmacokinetics in the blood of rats; Los Alamos National Laboratory: Los Alamos, July 1994, 1994). These limitations could conceivably be circumvented by liposomal encapsulation and preferential delivery to tumor tissue, but efficiently loading staurosporine or its analogues into liposomes has thus far not been feasible (Yamauchi, M. et al., Biol Pharm Bull 2005, 28 (7), 1259-64).

Staurosporine avidly targets the PKC family of signaling proteins in addition to other kinases such as PKA and PKG, which play a key role in tumorigenesis (da Rocha, A. B. et al., Oncologist 2002, 7 (1), 17-33; Sato, W. et al., Biochem Biophys Res Commun 1990, 173 (3), 1252-7; Satake, N. et al., Gen Pharmacol 1996, 27 (4), 701-5). Staurosporine treatment has been proposed for glioblastoma, a lethal cancer for which current treatments are of limited benefit and have serious toxicity (Wen, P. Y. et al., N Engl J Med 2008, 359 (5), 492-507; Stupp, R. et al., J Clin Oncol 2007, 25 (26), 4127-36). However, high staurosporine doses would be required to exceed plasma al-acid glycoprotein (hAGP) binding effects and allow sufficient free drug for antitumor activity (Fuse, E. et al., Cancer Res 1998, 58 (15), 3248-53). This level of dosing would cause unacceptable toxicity from pan kinase inhibition in normal tissues.

A potential solution to the obstacles hindering further development of staurosporine and its analogues is offered by liposomal encapsulation because: First, liposomes offer improved circulation half-life by shielding payload from plasma hAGP proteins, and they slow hepatic-renal clearance due to optimal sizing combined with PEGylation; and secondly, leaky microvasculature at tumors and metastases facilitates preferential delivery of liposomal payload to tumor tissue, a selective effect that significantly bypasses the blood brain barrier (BBB) and which can be enhanced by tumor cell/vessel targeting of the carrier liposomes (Wang, A. Z. et al., Nanoparticle Delivery of Cancer Drugs. Annu Rev Med 2011; Simberg, D. et al., Biomaterials 2009, 30 (23-24), 3926-33).

Various liposomal remote loading methods incorporating chemical and pH gradients have been developed to encapsulate doxorubicin, topotecan and irinotecan (Drummond, D. C. et al., Cancer Res 2006, 66 (6), 3271-7; Sadzuka, Y. et al., J Control Release 2005, 108 (2-3), 453-9). However, staurosporine encapsulation by liposomes has been poor when attempted with these methodologies (Hashimoto, K. et al., Endocrinol Jpn 1976, 23 (3), 243-9; Yamauchi, M. et al., Int J Pharm 2008, 351 (1-2), 250-8).

Therefore, there is a need in the art for the development of methods of producing liposomal drug compositions, wherein the drug (e.g. staurosporine) is stably and efficiently encapsulated and wherein the liposome is efficiently delivered to the drug's specific site of action (e.g. tumor). The present invention as provided herein cures these and other needs in the art by providing, inter alia, methods of making liposomally encapsulated drugs (e.g. staurosporine) and compositions related thereto.

### BRIEF SUMMARY OF THE INVENTION

The present invention provided herein relates to, inter alia, novel methods of making liposomally encapsulated drugs using reverse pH gradients and optimizing internal buffer compositions. Further provided herein are liposome compositions including an active pharmaceutical ingredient (e.g. staurosporine) and uses thereof to treat a variety of disease (e.g. cancer, inflammatory, neurological and cardiovascular diseases).

In one aspect, a method of forming a liposomally encapsulated drug is provided. The method includes contacting an unloaded liposome with a drug in an exterior aqueous medium at an exterior aqueous medium pH, wherein the unloaded liposome includes an interior cavity aqueous medium with an interior cavity pH at least 2 units higher than the exterior aqueous medium pH. The drug is allowed to move from the exterior aqueous medium to the interior cavity thereby forming a liposomally encapsulated drug.

In another aspect, a liposome including an interior cavity with a staurosporine phosphate or staurosporine sulfate and an interior cavity aqueous medium is provided.

In another aspect, a pharmaceutical composition prepared according to the methods provided herein including embodiments thereof is provided.

In another aspect, a method of treating a disease in a subject in need thereof is provided. The method includes administering to the subject a therapeutically effective amount of a pharmaceutical composition prepared according to the methods provided herein including embodiments thereof.

### BRIEF DESCRIPTION OF THE DRAWINGS

FIG. 1A and FIG. 1B. Liposomes containing staurosporine were of favorable size and charge. FIG. 1A: Size (diameter, nm) of the staurosporine liposomes measured by differential light scattering (DLS). FIG. 1B: Representative scanning electron microscope (SEM) images showing the spherical structure, integrity and diameter of the staurosporine liposomes. The size bar in the right of the image is 100 nm.

FIG. 2A-2C. The effect of pH on staurosporine encapsulation efficiency. FIG. 2A: The encapsulation efficiency with the internal buffer pH at 3 and at 7.4 is compared with the external buffer constant at pH=3. When a gradient existed encapsulation was increased by 14 fold. FIG. 2B: The effect of different internal buffers on encapsulation when the internal pH was 7.4 and external was 3. Ammonium phosphate 300 mM was associated with the highest measured encapsulation efficiency, while ammonium sulfate was close. Sodium salt buffers led to poor encapsulation. FIG. 2C: Effect of external buffer pH when the internal buffer was held constant at pH 7.4. When the external buffer, either HEPES or PBS, was pH 3, encapsulation efficiency approached 70%.

FIG. 3: Staurosporine encapsulation efficiency according to initial drug to lipid ratio (mole/mole). Staurosporine was loaded into liposomes using the reverse pH gradient method. The bars represent mean encapsulation efficacies calculated from 3 samples, and it can be seen that an optimal drug/lipid ratio occurs in terms of encapsulation efficiency at 0.09 (mole/mole).

FIG. 4A and FIG. 4B. Staurosporine was retained within liposomes incubated in vitro with (FIG. 4A) PBS and (FIG. 4 B) human serum. Retention was measured after incubation in serum at room temperature for a range of times. Staurosporine liposomes were separated from the incubation medium by column chromatography, the staurosporine measured spectrophotometrically for liposomes incubated in PBS and with HPLC for those incubated in serum. Virtually all the staurosporine was contained within the liposomes at 3 hours of incubation in PBS or serum, with approximately 80% of the staurosporine remaining after 6 hours of incubation.

FIG. 5A-5F: In vitro cytoxocity of liposomally encapsulated staurosporine. The MTT Cell viability assay of staurosporine liposomes and free staurosporine was performed with human glioblastoma cell lines, both established and freshly derived from surgical isolates. The experiments were carried out in triplicate with data represented as the mean±SEM. Each histogram depicts a different cell line: FIG. 5A shows results for cell line A172, FIG. 5B shows results for cell line U251, FIG. 5C shows results for cell line U118, FIG. 5D shows results for cell line U87, FIG. 5E shows results for the freshly derived line GBM4 and FIG. 5F shows results for the freshly derived line GBM8. EC50 values are indicated for encapsulated and free staurosporine.

FIG. 6A-6C. Encapsulated staurosporine at a low dose shows in vivo antitumor efficacy with tumor xenograft models. FIG. 6A: U87 glioblastoma cells were implanted s.c. in nude mice. When tumors were established and had reached volumes of 40-50 mm3 the mice were sorted into a treatment and a control group which had the same mean tumor volume. The treatments were then initiated: control (♦), staurosporine liposomes (▪) was injected at 1 mg/kg/dose i.v three times per week for three weeks, x-axis represents days after tumor implantation. The data show that the encapsulated staurosporine was not removed from the circulation before it could exert a robust antitumor effect. FIG. 6B: After three weeks staurosporine treated mice were not treated for 5 weeks. Tumors were reestablished in those mice and after reaching 120 mm³ treatments were administered: control (♦), staurosporine liposomes (▪) was injected at 1 mg/kg/dose i.v three times a week for one week, the x-axis represents days after starting the treatment. Even with limited dosing during the first post-growth week, very large tumors, and a two week observation period, the staurosporine liposomes still exerted a clear antitumor effect. FIG. 6C: Liposomal staurosporine had no effect on body weight. Mean body weight for the mouse groups treated with PBS or encapsulated staurosporine before and after the first round of treatment is shown. The bars are means with standard error of the mean (SEM). Unpaired, parametric t-test comparisons indicated no significant difference between weights taken before versus after treatment (PBS p=0.58; Liposomal staurosporine p=0.87).

FIG. 7. Efficiency of drug encapsulation for other drug chemotypes.

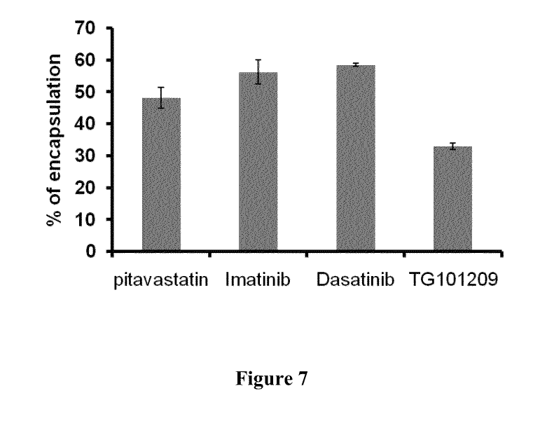

FIG. 8A-8C. In vitro Activity of encapsulated staurosporine against broad range of human cancer cell lines. The cytotoxic effect of liposomal staurosporine and free staurosporine on established human cancer cell lines PC3 pancreatic (FIG. 8B) and MDAMB231 (FIG. 8A) breast cancer cells and mouse melanoma cell line B16F10 (FIG. 8C) was evaluated. The results show that the EC50 of both liposomal and free staurosporine these lines were in between 10-50 nm (FIG. 8A-8C).

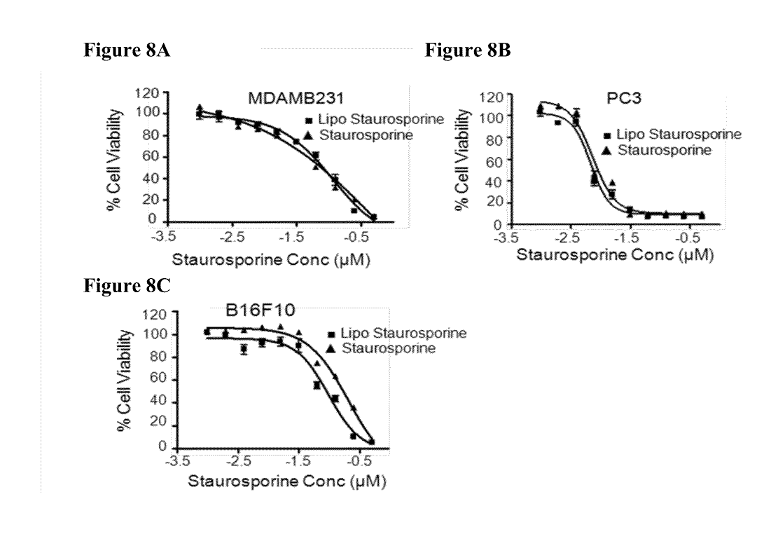

FIG. 9A-9D. In vivo anti-tumor activity of encapsulated staurosporine. Athymic nu/nu mice (5-6 weeks old) were subcutaneously inoculated in the right and left flanks with 2 million U87 cells. The tumors were allowed to grow very large 180-200 mm³ before treatment was started, treatment with liposomal staurosporine over two weeks markedly slowed the growth of the tumors relative to those treated by PBS and free staurosporine (FIG. 9A and FIG. 9B). The photographs in FIG. 9B show a very consistent size reduction in treated tumors with very little variance, indicating a real, substantial, and statistically significant effect (p<0.004), and revealing that free staurosporine had no demonstrable effect. However, free staurosporine was toxic, as body weight did decline, while liposomal staurosporine and PBS-treated animals had no weight difference (FIG. 9C). Ki-67 staining in tumor revealed that tumor proliferation was significantly reduced with liposomal staurosporine treatment (p<0.03; FIG. 9D).

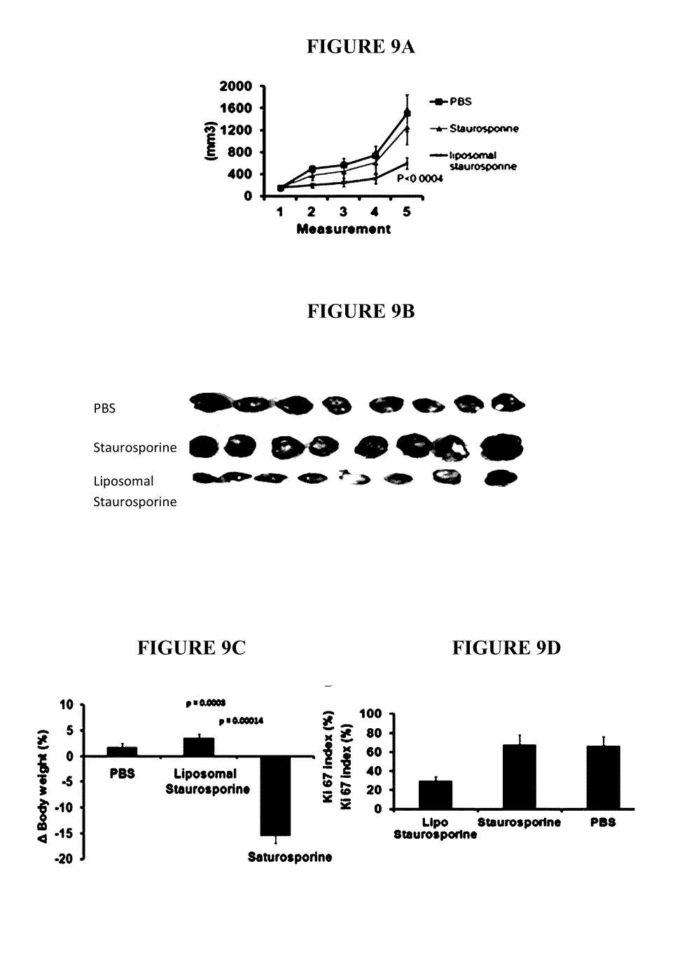

FIG. 10. Drug encapsulation assay. By using the reverse pH gradient technology of the present invention LY29004 (PI3K inhibitor) and Gefitinib (EGFR inhibitor) are encapsulated with good efficacy.

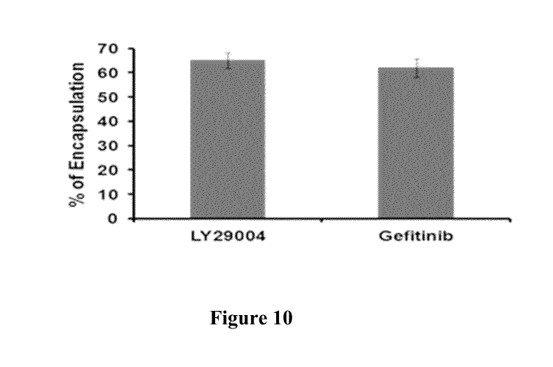

### DETAILED DESCRIPTION OF THE INVENTION

Provided herein are methods of compositions of efficiently encapsulating therapeutically effective amounts of an active pharmaceutical ingredient (e.g. staurosporine) in a liposome using pH gradient reversal and optimizing the internal buffer conditions of the liposome. Further provided herein are methods of treating a variety of disease using these very effective liposomal compositions. Examples of diseases that can be treated with the methods and compositions provided herein and embodiments thereof are without limitation, cancer, inflammatory diseases, neurological diseases and cardiovascular diseases.

### I. DEFINITIONS

The abbreviations used herein have their conventional meaning within the chemical and biological arts.

The terms “a,” “an,” or “the” as used herein not only include aspects with one member, but also aspects with more than one member. For example, an embodiment including “a buffer and a chelating agent” should be understood to present aspects with at least a second buffer, at least a second chelating agent, or both.

The term “or” as used herein should in general be construed non-exclusively. For example, an embodiment of “a formulation including A or B” would typically present an aspect with a formulation including both A and B. “Or” should, however, be construed to exclude those aspects presented that cannot be combined without contradiction (e.g., a formulation pH that is between 9 and 10 or between 7 and 8).

“Formulation,” “composition,” and “preparation” as used herein are equivalent terms referring to a composition of matter suitable for pharmaceutical use (i.e., producing a therapeutic effect as well as possessing acceptable pharmacokinetic and toxicological properties).

As used herein, the term “pharmaceutically” acceptable is used as equivalent to physiologically acceptable. In certain embodiments, a pharmaceutically acceptable composition or preparation will include agents for buffering and preservation in storage, and can include buffers and carriers for appropriate delivery, depending on the route of administration.

The terms “drug”, “active pharmaceutical ingredient”, “API” and the like refer to the active ingredient of a drug product. An API is typically a chemical substance or mixture of chemical substances. Such substances are intended to furnish pharmacological activity or other direct effect in the diagnosis, cure, mitigation, treatment or prevention of disease or to affect the structure and function of the body of a subject. “Drug product” refers, in the customary sense, to a composition useful in the diagnosis, cure, mitigation, treatment or prevention of a disease or disorder in the healing arts, e.g., medical, veterinary, and the like. Further to any aspect disclosed herein, in some embodiments the composition is a pharmaceutical composition suitable for use as a drug product.

As used herein, the terms “prevent” and “treat” are not intended to be absolute terms. Treatment can refer to any delay in onset, e.g., reduction in the frequency or severity of symptoms, amelioration of symptoms, improvement in patient comfort, and the like. The effect of treatment can be compared to an individual or pool of individuals not receiving a given treatment, or to the same patient before, or after cessation of, treatment.

The terms “subject,” “patient,” “individual,” and the like as used herein are not intended to be limiting and can be generally interchanged. That is, an individual described as a “patient” does not necessarily have a given disease, but may be merely seeking medical advice. The term “subject” as used herein includes all members of the animal kingdom prone to suffering from the indicated disorder. In some aspects, the subject is a mammal, and in some aspects, the subject is a human.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “disease” refers to any deviation from the normal health of a mammal and includes a state when disease symptoms are present, as well as conditions in which a deviation (e.g., infection, gene mutation, genetic defect, etc.) has occurred, but symptoms are not yet manifested. According to the present invention, the methods disclosed herein are suitable for use in a patient that is a member of the Vertebrate class, Mammalia, including, without limitation, primates, livestock and domestic pets (e.g., a companion animal). Typically, a patient will be a human patient.

An “effective amount” of a compound is an amount sufficient to contribute to the treatment, prevention, or reduction of a symptom or symptoms of a disease. Where recited in reference to a disease treatment, an “effective amount” may also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) a disease, disorder or condition, or reducing the likelihood of the onset (or reoccurrence) of a disease, disorder or condition or symptoms thereof. The full prophylactic effect does not necessarily occur by administration of one dose, and may occur only after administration of a series of doses. Thus, a prophylactically effective amount may be administered in one or more administrations.

The terms “about” and “approximately equal” are used herein to modify a numerical value and indicate a defined range around that value. If “X” were the value, “about X” or “approximately equal to X” would generally indicate a value from 0.90X to 1.10X. Any reference to “about X” minimally indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to disclose, e.g., “0.98X.” When “about” is applied to the beginning of a numerical range, it applies to both ends of the range. Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.”

As used herein, the terms “administer,” “administered,” or “administering” refer to methods of administering the liposome compositions of the present invention. The liposome compositions of the present invention can be administered in a variety of ways, including topically, parenterally, intravenously, intradermally, intramuscularly, colonically, rectally or intraperitoneally. The liposome compositions can also be administered as part of a composition or formulation.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine. and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the phrase “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids. Non-limiting examples of base salts include ammonium salts, alkali metal salts, such as sodium and potassium salts; alkaline earth metal salts, such as calcium and magnesium salts; salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others.

As used herein, the term “liposome” encompasses any compartment enclosed by a lipid bilayer. The term liposome includes unilamellar vesicles which are comprised of a single lipid bilayer and generally have a diameter in the range of about 20 to about 400 nm. Liposomes can also be multilamellar, which generally have a diameter in the range of 1 to 10 μm. In some embodiments, liposomes can include multilamellar vesicles (MLV), large unilamellar vesicles (LUV), and small unilamellar vesicles (SUV).

As used herein, the term “lipid” refers to lipid molecules that can include fats, waxes, steroids, cholesterol, fat-soluble vitamins, monoglycerides, diglycerides, phospholipids, sphingolipids, glycolipids, cationic or anionic lipids, derivatized lipids, and the like, as described in detail below. Lipids can form micelles, monolayers, and bilayer membranes. The lipids can self-assemble into liposomes.

### II. LIPOSOMES

The liposomes of the present invention comprise an aqueous compartment enclosed by at least one lipid bilayer. The aqueous compartment enclosed by a lipid bilayer is referred to herein as an “interior cavity.” The aqueous compartment enclosed by the lipid bilayer is referred to herein as an “interior cavity aqueous medium.” When lipids that include a hydrophilic headgroup are dispersed in water they can spontaneously form bilayer membranes referred to as lamellae. The lamellae are composed of two monolayer sheets of lipid molecules with their non-polar (hydrophobic) surfaces facing each other and their polar (hydrophilic) surfaces facing the aqueous medium. The term liposome includes unilamellar vesicles which are comprised of a single lipid bilayer and generally have a diameter in the range of about 20 to about 400 nm, about 50 to about 300 nm, or about 100 to 200 nm. In some embodiments, the liposome has a size from about 20 nm to about 400 nm, from about 40 nm to about 400 nm, from about 60 nm to about 400 nm, from about 80 nm to about 400 nm, from about 100 nm to about 400 nm, from about 120 nm to about 400 nm, from about 140 nm to about 400 nm, from about 160 nm to about 400 nm, from about 180 nm to about 400 nm, from about 200 nm to about 400 nm, from about 220 nm to about 400 nm, from about 240 nm to about 400 nm, from about 260 nm to about 400 nm, from about 280 nm to about 400 nm, from about 300 nm to about 400 nm, from about 320 nm to about 400 nm, from about 340 nm to about 400 nm, from about 360 nm to about 400 nm, or from about 380 nm to about 400 nm. In some embodiments, the liposome has a size of approximately 20, 40, 60, 80, 100, 120, 140, 160, 180, 200, 220, 240, 260, 280, 300, 320, 340, 360, 380, or 400 nm.

Liposomes can also be multilamellar having a diameter in the range of approximately 1 to approximately 10 μm. Multilamellar liposomes may consist of several (anywhere from two to hundreds) unilamellar vesicles forming one inside the other in diminishing size, creating a multilamellar structure of concentric phospholipid spheres separated by layers of water. Alternatively, multilamellar liposomes may consist of many smaller non concentric spheres of lipid inside a large liposome. In some embodiments, the liposome has a size from about 1 μm to about 10 μm, from about 2 μm to about 10 μm, from about 3 μm to about 10 μm, from about 4 μm to about 10 μm, from about 5 μm to about 10 μm, from about 6 μm to about 10 μm, from about 7 μm to about 10 μm, from about 8 μm to about 10 μm, or from about 9 μm to about 10 μm. In some embodiment, the liposome has a size of approximately 1, 2, 3, 4, 5, 6, 7, 8, 9 or 10 μm.

The liposomes of the present invention can contain any suitable lipid, including cationic lipids, zwitterionic lipids, neutral lipids, or anionic lipids. Suitable lipids can include fats, waxes, steroids, cholesterol, fat-soluble vitamins, monoglycerides, diglycerides, phospholipids, sphingolipids, glycolipids, cationic or anionic lipids, derivatized lipids, and the like.

Suitable phospholipids include but are not limited to phosphatidylcholine (PC), phosphatidic acid (PA), phosphatidylethanolamine (PE), phosphatidylglycerol (PG), phosphatidylserine (PS), and phosphatidylinositol (PI), dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin. In some embodiments, the phospholipid is DOPE. In other embodiments, the phospholipid is DSPC. Lipid extracts, such as egg PC, heart extract, brain extract, liver extract, and soy PC, are also useful in the present invention. In some embodiments, soy PC can include Hydro Soy PC (HSPC). In certain embodiments, the lipids can include derivatized lipids, such as PEGylated lipids. Derivatized lipids can include, for example, DSPE-PEG2000, cholesterol-PEG2000, DSPE-polyglycerol, or other derivatives generally known in the art. In some embodiments, the derivatized phospholipid is DSPE-PEG2000.

Cationic lipids contain positively charged functional groups under physiological conditions. Cationic lipids include, but are not limited to, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N-[1-(2,3,-ditetradecyloxy)propyl]-N,N-dimethyl-N-hydroxyethylammonium bromide (DMRIE), N-[1-(2,3,dioleyloxy)propyl]-N,N-dimethyl-N-hydroxy ethylammonium bromide (DORIE), 3β-[N—(N′,N′-dimethylaminoethane) carbamoyl]cholesterol (DC-Chol), dimethyldioctadecylammonium (DDAB) and N,N-dimethyl-2,3-dioleyloxy)propylamine (DODMA).

Liposomes of the present invention may contain steroids, characterized by the presence of a fused, tetracyclic gonane ring system. Examples of steroids include, but are not limited to, cholesterol, cholic acid, progesterone, cortisone, aldosterone, estradiol, testosterone, dehydroepiandrosterone. Synthetic steroids and derivatives thereof are also contemplated for use in the present invention. In some embodiments the steroid is cholesterol.

In some embodiments, the liposome includes one or more lipids which can be a phospholipid, a steroid, and/or a cationic lipid. In some embodiments, the phospholipid is a phophatidylcholine, a phosphatidylglycerol, a phosphatidylethanolamine, a phosphatidylserine, a phosphatidylinositol, or a phosphatidic acid. In some embodiments, the phosphatidylcholine is distearoyl phosphatidyl choline (DSPC). In some embodiments, the phosphatidylethanolamine is dioleoyl phosphatidyl ethanolamine (DOPE). In some embodiments, the phosphatidylethanolamine is distearoyl-phosphatidyl-ethanolamine (DSPE). In some embodiments, the phosphatidylethanolamine is derivatized. In some further embodiments, the derivatized phosphatidylethanolamine is DSPE-PEG (2000). In some embodiments, the steroid is cholesterol.

The liposome of the present invention may include about ten or fewer types of lipids, or about five or fewer types of lipids, or about three or fewer types of lipids. In some embodiment, the liposome includes four lipids. In some further embodiment, the lipids are cholesterol, DOPE, DSPC, and DSPE-PEG (2000). In some further embodiment, the molar ratio of cholesterol, DOPE, DSPC, and DSPE-PEG (2000) is 6:6:6:1.

Any suitable combination of lipids can be used to provide the liposomes of the invention. The lipid compositions can be tailored to affect characteristics such as leakage rates, stability, particle size, zeta potential, protein binding, in vivo circulation, and/or accumulation in tissues or organs. For example, DSPC and/or cholesterol can be used to decrease leakage from liposomes. Negatively or positively charged lipids, such as DSPG and/or DOTAP, can be included to affect the surface charge of a liposome.

One of skill in the ordinary art will immediately recognize that the lipid compositions provided herein may be adjusted to modulate the release properties or other characteristics of the liposomes as required by a given application.

### III. METHODS OF DRUG ENCAPSULATION

The present invention relates to novel methods of making liposomally encapsulated drugs using reverse pH gradients and optimizing internal buffer compositions. In one aspect, a method of forming a liposomally encapsulated drug (e.g. staurosporine) is provided. The method includes contacting an unloaded liposome with a drug in an exterior aqueous medium at an exterior aqueous medium pH, wherein the unloaded liposome includes an interior cavity aqueous medium with an interior cavity pH at least 2 units higher than the exterior aqueous medium pH. The drug is allowed to move from the exterior aqueous medium to the interior cavity thereby forming a liposomally encapsulated drug.

An “unloaded liposome” as provided herein refers to a liposome (e.g. unilamellar, multilamellar liposome) which does not include an active pharamceutical ingredient (drug) in the interior cavity or otherwise attached or linked to the liposomal structure. The “interior cavity” of an unloaded liposome refers to the compartment enclosed by at least one lipid bilayer of the liposome and includes an interior cavity aqueous medium. The interior cavity aqueous medium may be an aqueous salt solution (e.g. an aqueous solution with a buffer), wherein the salt may be an ammonium phosphate, ammonium sulfate, sodium phosphate, or sodium sulfate. The interior cavity aqueous medium is composed such that an active pharamceutical ingredient (drug) is allowed to move from an exterior aqueous medium to the interior cavity of the liposome, thereby forming a liposomally encapsulated active pharmaceutical ingredient (drug). The interior cavity aqueous medium has a pH which faciliates the movement of the drug from the exterior aqueous medium to the interior cavity (e.g. by facilitating a pH gradient between the interior cavity and the exterior aqueous medium). In some embodiments, the interior cavity pH is at least 2 units (e.g, 2, 3, 4, 5, 6 units) higher than the exterior aqueous medium pH. In some embodiment, the interior cavity pH is from about 5 to about 9. In other embodiments, the interior cavity pH is from about 6 to about 8. In some embodiments, the interior cavity pH is from about 7 to about 8. In other embodiments, the interior cavity pH is from about 7.4 to about 7.6. In some embodiments, the interior cavity pH is from about 5 to about 9, from about 5.5 to about 9, from about 6 to about 9, from about 6.5 to about 9, from about 7 to about 9, from about 7.1 to about 9, from about 7.2 to about 9, from about 7.3 to about 9, from about 7.4 to about 9, from about 7.5 to about 9, from about 7.6 to about 9, from about 7.7 to about 9, from about 7.8 to about 9, from about 7.9 to about 9, from about 8 to about 9, or from about 8.5 to about 9. In some embodiments, the interior cavity pH is approximately about 5, 5.5, 6, 6.5, 7, 7.1, 7.2, 7.3, 7.4, 7.5, 7.6, 7.7, 7.8, 7.9, 8, 8.5, or 9.

An “exterior aqueous medium” as provided herein refers to an aqueous solution (e.g. an aqueous solution with a buffer) wherein a active pharmaceutical ingredient (drug) may be dissolved. Examples of an exterior aqueous medium useful for the invention provided herein include without limitation 4-(2-hydroxyethyl)-1-piperazineethanesulfonic acid HEPES and phosphate buffered saline (PBS). In some embodiments, the exterior aqueous medium pH is about 1 to 4. In other embodiments the exterior aqueous medium pH is about 2 to 4. In some embodiments, the exterior aqueous medium pH is about 2.5 to 3.5. In some embodiments, the exterior aqueous medium pH is about 3. In some embodiments, the exterior aqueous medium pH is about 1 to 4, is about 1.5 to 4, is about 2 to 4, is about 2.5 to 4, is about 3 to 4, is about 3.1 to 4, is about 3.2 to 4, is about 3.3 to 4, is about 3.4 to 4, is about 3.5 to 4, is about 3.6 to 4, is about 3.7 to 4, is about 3.9 to 4, or is about 3.9 to 4. In some embodiments, the exterior aqueous medium pH is about 1, 1.5, 2, 2.5, 3, 3.1, 3.2, 3.3, 3.4, 3.5, 3.6, 3.7, 3.8, 3.9, or 4.

In some embodiments, the drug is positively charged in the exterior aqueous medium pH. In some embodiments, the drug has a pKa at least 2 units higher than the exterior aqueous medium pH. In some embodiments, the pKa is at least 2.5 units higher than the exterior aqueous medium pH. In some embodiments, the pKa is at least 3 units higher than the exterior aqueous medium pH. In some embodiments, the pKa is at least 3.5 units higher than the exterior aqueous medium pH. In some embodiments, the pKa is at least 4 units higher than the exterior aqueous medium pH. In some embodiments, the pKa is at least 4.5 units higher than the exterior aqueous medium pH. In some embodiments, the pKa is at least 5 units higher than the exterior aqueous medium pH. In some embodiments, the pKa is at least 5.5 units higher than the exterior aqueous medium pH. In some embodiments, the pKa is at least 6 units higher than the exterior aqueous medium pH. In some embodiments, the pKa is at least 6.5 units higher than the exterior aqueous medium pH. In some embodiments, the pKa is at least 7 units higher than the exterior aqueous medium pH. In some embodiments, the pKa is at least 7.5 units higher than the exterior aqueous medium pH. In some embodiments, the pKa is at least 8 units higher than the exterior aqueous medium pH. In some embodiments, the pKa is at least 8.5 units higher than the exterior aqueous medium pH. In some embodiments, the pKa is at least 9 units higher than the exterior aqueous medium pH. In some embodiments, the pKa is at least 10 units higher than the exterior aqueous medium pH. In some embodiments, the pKa is at least 10.5 units higher than the exterior aqueous medium pH. In some embodiments, the pKa is at least 11 units higher than the exterior aqueous medium pH. In some embodiments, the pKa is about 5 and the exterior aqueous medium pH is about 3.

For the methods and compositions provided herein the drug may be present in the exterior aqueous medium as an exterior aqueous medium drug salt. The drug may further be present in the interior cavity of the liposome as an interior cavity drug salt. The exterior aqueous medium drug salt may be citrate. The interior cavity drug salt may be a sulfate or a phosphate. In some embodiments, the drug is present in the exterior aqueous medium as an exterior aqueous medium drug salt and is present in the interior cavity as an interior cavity drug salt. In some embodiments, the exterior aqueous medium drug salt is citrate. In other embodiments, the interior cavity drug salt is phosphate or sulfate. In other embodiments, the interior cavity drug salt is phosphate. In other embodiments, the interior cavity drug salt is sulfate.

Active pharmaceutical ingredients contemplated for the methods and compositions provided herein include without limitation compounds and small molecules useful for the treatment of cancer (e.g. staurosporine), cholesterol diseases (e.g. statins), inflammatory diseases, neurological diseases and cardiovascular diseases.

The active pharmaceutical ingredient (drug) useful for the methods and compositions provided herein including embodiments thereof may be a cancer drug. Examples of cancer drugs include without limitation protein kinase inhibitors (e.g. tyrosine kinase inhibitors) and chemotherapeutics (e.g. doxorubicin). In some embodiments, the drug is a chemotherapeutic drug. In other embodiments, the drug is a protein kinase inhibitor.

For the methods provided herein including embodiments thereof the drug may be staurosporine. Thus, in some embodiments, the drug is staurosporine. Staurosporine as provided herein is a natural alkaloid having the systematic IUPAC name: (9S,10R,11R,13R)-2,3,10,11,12,13-Hexahydro-10-methoxy-9-methyl-11-(methylamino)-9,13-epoxy-1H,9H-diindolo[1,2,3-gh:3′,2′,1′-1m]pyrrolo[3,4-j][1,7]benzodiazonin-1-one. In the customary sense, staurosporine refers to CAS Registry No. 62996-74-1. In some embodiments, the aqueous medium drug salt is staurosporine citrate and the interior cavity drug salt is staurosporine phosphate or staurosporine sulfate.

For the methods provided herein including embodiments thereof the active pharmaceutical ingredient (drug) may be doxorubicin. Thus, in some embodiments, the drug is doxorubicin. Doxorubicin as provided herein is an anthracycline antibiotic having the systematic IUPAC name: (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione. In the customary sense, doxorubicin refers to CAS Registry No. 23214-92-8. In some embodiments, the aqueous medium drug salt is doxorubicin citrate and the interior cavity drug salt is doxorubicin phosphate or doxorubicin sulfate.

For the methods provided herein including embodiments thereof the active pharmaceutical ingredient (drug) may be dasatinib. Thus, in some embodiments, the drug is dasatinib. Dasatinib as provided herein is a tyrosine kinase inhibitor having the systemic IUPAC name: N-(2-chloro-6-methylphenyl)-2-[[6-[4-(2-hydroxyethyl)-1-piperazinyl]-2-methyl-4-pyrimidinyl]amino]-5-thiazole carboxamide monohydrate. In the customary sense, dasatinib refers to CAS Registry No. 302962-49-8. In some embodiments, the aqueous medium drug salt is dasatinib citrate and the interior cavity drug salt is dasatinib phosphate or dasatinib sulfate.

For the methods provided herein including embodiments thereof the active pharmaceutical ingredient (drug) may be imatinib. Thus, in some embodiments, the drug is imatinib. Imatinib as provided herein is a tyrosine kinase inhibitor having the systemic IUPAC name: 4-[(4-methylpiperazin-1-yl)methyl]-N-(4-methyl-3-{[4-(pyridin-3-yl)pyrimidin-2-yl]amino}phenyl)benzamide. In the customary sense, imatinib refers to CAS Registry No. 152459-95-5. In some embodiments, the aqueous medium drug salt is imatinib citrate and the interior cavity drug salt is imatinib phosphate or imatinib sulfate.

For the methods provided herein including embodiments thereof the active pharmaceutical ingredient (drug) may be gefitinib. Thus, in some embodiments, the drug is gefitinib. Gefitinib as provided herein is a tyrosine kinase inhibitor having the systemic IUPAC name: N-(3-chloro-4-fluoro-phenyl)-7-methoxy-6-(3-morpholin-4-ylpropoxy)quinazolin-4-amine. In the customary sense, gefitinib refers to CAS Registry No. 184475-35-2. In some embodiments, the aqueous medium drug salt is gefitinib citrate and the interior cavity drug salt is gefitinib phosphate or gefitinib sulfate.

For the methods provided herein including embodiments thereof the active pharmaceutical ingredient (drug) may be LY 29004. Thus, in some embodiments, the drug is LY 29004. LY 29004 as provided herein is an inhibitor of phosphatidylinositol 3 (PI3) kinase having the structure of formula

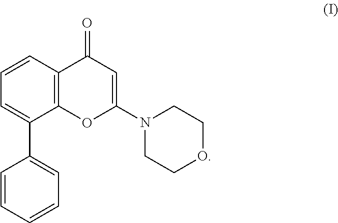 [SMILES: O=c1cc(N2CCOCC2)oc2c(-c3ccccc3)cccc12]

In some embodiments, the aqueous medium drug salt is LY 29004 citrate and the interior cavity drug salt is LY 29004 phosphate or LY 29004 sulfate.

For the methods provided herein including embodiments thereof the active pharmaceutical ingredient (drug) may be TG101209. Thus, in some embodiments, the drug is TG101209. TG101209 as provided herein is a JAK2-selective kinase inhibitor having the structure of formula:

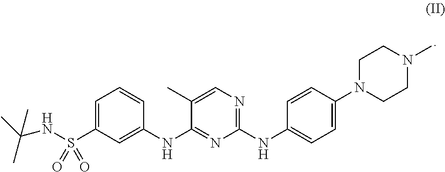 [SMILES: Cc1cnc(Nc2ccc(N3CCN(C)CC3)cc2)nc1Nc1cccc(S(=O)(=O)NC(C)(C)C)c1]

In some embodiments, the aqueous medium drug salt is TG101209 citrate and the interior cavity drug salt is TG101209 phosphate or TG101209 sulfate.

The active pharmaceutical ingredient (drug) useful for the methods and compositions provided herein including embodiments thereof may be a cholesterol drug. A cholesterol drug as provided herein refers to a drug having cholesterol lowering ability. A nonlimiting example of a cholesterol drugs are statins. For the methods provided herein including embodiments thereof the active pharmaceutical ingredient (drug) may be a statin. Thus, in some embodiments, the drug is a statin. In some further embodiment, the drug is pitavastatin. Pitavastatin as provided herein is a tyrosine kinase inhibitor having the systemic IUPAC name: (3R,5S,6E)-7-[2-cyclopropyl-4-(4-fluorophenyl)quinolin-3-yl]-3,5-dihydroxyhept-6-enoic acid. In the customary sense, pitavastatin refers to CAS Registry No. 147511-69-1. In some embodiments, the aqueous medium drug salt is pitavastatin citrate and the interior cavity drug salt is pitavastatin phosphate or pitavastatin sulfate.

In the methods provided herein including embodiments thereof a liposomally incapsulated drug is formed by allowing a drug to move from the exterior aqueous medium surrounding a liposome to the interior cavity of the liposome. The drug may be loaded into the liposome by imposing a pH gradient across the liposome membrane, such that the liposome interior (i.e. the interior cavity aqueous medium) is more basic than the aqueous medium surrounding the liposome (i.e. the exterior aqueous medium). The exterior aqueous medium contains the drug as exterior aqueous medium drug salt (e.g. citrate) and has a low pH (e.g. about 1-5). Further, the pKa of the exterior aqueous medium drug salt may be at least 2 units higher than the exterior aqueous medium pH. The exterior aqueous medium drug salt may exist in a charged form, have good solubility at a pH of less than 5 and permeate (e.g. rapidly permeate) across the liposome membrane. Upon reaching the more basic interior cavity aqueous medium, the active pharmaceutical ingredient may be present as an interior cavity drug salt. The interior cavity drug salt may have lower (e.g. low solubility at a pH greater than 6). The interior drug salt may be a charged membrane-impermeable form of the drug thereby driving the continued uptake and retention of the drug in the interior cavity of the liposome. Thus, a concentration gradient may be formed between the exterior aqueous medium containing the highly soluble form of the drug and the interior cavity containing the less soluble form of the drug. In some embodiments, the solubility of the interior cavity aqueous medium drug salt is sufficiently low such that the drug salt precipitates within the interior cavity aqueous medium but not in the exterior aqueous medium. In other embodiments, the interior cavity aqueous medium drug salt forms crystals in the interior cavity of the liposome. Thus, in some embodiments, the exterior aqueous medium drug salt is more soluble in the exterior aqueous medium than the interior cavity drug salt in the interior cavity aqueous medium. In some embodiments, the interior cavity drug salt is in crystalline form and the exterior aqueous medium drug salt is solubilized in the exterior aqueous medium.

The step of allowing a drug to move from the exterior aqueous medium surrounding a liposome to the interior cavity of the liposome as provided herein may be performed at a temperature above room temperature for a certain amount of time. In some embodiments, the allowing is performed at about 50° C. The allowing may be performed at about 50° C. for at least 15 minutes. In some embodiments, the allowing is performed for about 20 minutes to about 60 minutes. In some embodiments, the allowing is performed for about 30 minutes to about 60 minutes. In other embodiments, the allowing is performed for about 40 minutes to about 60 minutes. In some embodiments, the allowing is performed for about 50 minutes to about 60 minutes.

In order to provide for efficient loading of the liposome with pharmaceutically effective amounts of a drug, the molar ratio of active pharmaceutical ingredient (e.g. staurosporine) and lipid (unloaded liposome particles) may be 0.09. In some embodiments, the unloaded liposome and the drug are present at a ratio of at least 0.06 mol of liposome/mol of drug. In some embodiments, the unloaded liposome and the drug are present at a ratio of about 0.07 mol of liposome/mol of drug. In some embodiments, the unloaded liposome and the drug are present at a ratio of about 0.08 mol of liposome/mol of drug. In some embodiments, the unloaded liposome and the drug are present at a ratio of about 0.09 mol of liposome/mol of drug. In some embodiments, the unloaded liposome and the drug are present at a ratio of about 0.10 mol of liposome/mol of drug. In some embodiments, the unloaded liposome and the drug are present at a ratio of about 0.11 mol of liposome/mol of drug. In some embodiments, the unloaded liposome and the drug are present at a ratio of about 0.12 mol of liposome/mol of drug. In some embodiments, the unloaded liposome and the drug are present at a ratio of about 0.13 mol of liposome/mol of drug. In some embodiments, the unloaded liposome and the drug are present at a ratio of about 0.14 mol of liposome/mol of drug. In some embodiments, the unloaded liposome and the drug are present at a ratio of about 0.15 mol of liposome/mol of drug. In some embodiments, the unloaded liposome and the drug are present at a ratio of about 0.16 mol of liposome/mol of drug. In some embodiments, the unloaded liposome and the drug are present at a ratio of about 0.17 mol of liposome/mol of drug. In some embodiments, the unloaded liposome and the drug are present at a ratio of about 0.18 mol of liposome/mol of drug. In some embodiments, the unloaded liposome and the drug are present at a ratio of about 0.19 mol of liposome/mol of drug. In some embodiments, the unloaded liposome and the drug are present at a ratio of about 0.20 mol of liposome/mol of drug.

### IV. LIPOSOMAL COMPOSITIONS

Further, provided herein are liposomal compositions including an active pharmaceutical ingredient (e.g. staurosporine) and uses thereof to treat a variety of diseases (e.g. cancer, cholesterol, inflammatory, neurological and cardiovascular diseases). The liposomes provided herein may include an interior cavity with a salt of an active pharmaceutical ingredient (interior cavity drug salt) and an interior cavity aqueous medium. As described above the interior cavity drug salt provided herein may be more soluble at a low pH and is less soluble at a high pH. In one embodiment, the term low pH refers to a pH of less than 5 and the term high pH is a pH greater than 6. Where the pH of the interior cavity aqueous medium (i.e. interior cavity pH) is above 6, the interior cavity drug salt may be present in its soluble or its precipitated form. Thus, in one aspect a liposome including an interior cavity with an interior cavity drug salt and interior cavity aqueous medium is provided. In some embodiments, the interior cavity pH is greater than 6. In some further embodiment, the interior cavity pH is about 7 to 8. In some further embodiment, the interior cavity pH is about 7.4 to 7.6. In some further embodiments, the interior cavity drug salt is present in its soluble form. In some further embodiments, the interior cavity drug salt is present in its soluble and its precipitated form. In some further embodiments, the interior cavity drug salt is present in its soluble form. In some other further embodiment, the interior cavity drug salt is present in its crystallized form.

The active pharmaceutical ingredient (drug) as provided herein may be a cancer drug, a cholesterol drug, an inflammatory drug, a neurological drug or a cardiovascular drug. In some embodiments, the interior cavity drug salt is a phosphate or a sulfate. As described above the active pharmaceutical ingredient may be present as an interior cavity drug salt. In some embodiments, the interior cavity drug salt is a phosphate. In some embodiments, the interior cavity drug salt is a sulfate. In other embodiments, the drug is staurosporine. In some embodiments, the drug is doxorubicin. In other embodiments, the drug is dasatinib. In some embodiments, the drug is imatinib. In other embodiments, the drug is LY 29004. In other embodiments, the drug is gefitinib. In some embodiments, the drug is pitavastatin. In other embodiments, the drug is TG101209.

In one aspect, a liposome including an interior cavity with a staurosporine phosphate or staurosporine sulfate and an interior cavity aqueous medium is provided. In some embodiments, the liposome includes an interior cavity aqueous medium with an interior cavity pH of about 6 to 8. In other embodiments, the interior cavity pH is about 7 to 8. In other embodiments, the interior cavity pH is about 7.4 to 7.6. In some embodiments, the staurosporine phosphate or staurosporine sulfate is present at a therapeutically effective amount.

In one aspect, a liposome including an interior cavity with a doxorubicin phosphate or doxorubicin sulfate and an interior cavity aqueous medium is provided. In some embodiments, the liposome includes an interior cavity aqueous medium with an interior cavity pH of about 6 to 8. In other embodiments, the interior cavity pH is about 7 to 8. In other embodiments, the interior cavity pH is about 7.4 to 7.6. In some embodiments, the doxorubicin phosphate or doxorubicin sulfate is present at a therapeutically effective amount.

In one aspect, a liposome including an interior cavity with a dasatinib phosphate or dasatinib sulfate and an interior cavity aqueous medium is provided. In some embodiments, the liposome includes an interior cavity aqueous medium with an interior cavity pH of about 6 to 8. In other embodiments, the interior cavity pH is about 7 to 8. In other embodiments, the interior cavity pH is about 7.4 to 7.6. In some embodiments, the dasatinib phosphate or dasatinib sulfate is present at a therapeutically effective amount.

In one aspect, a liposome including an interior cavity with a imatinib phosphate or imatinib sulfate and an interior cavity aqueous medium is provided. In some embodiments, the liposome includes an interior cavity aqueous medium with an interior cavity pH of about 6 to 8. In other embodiments, the interior cavity pH is about 7 to 8. In other embodiments, the interior cavity pH is about 7.4 to 7.6. In some embodiments, the imatinib phosphate or imatinib sulfate is present at a therapeutically effective amount.

In one aspect, a liposome including an interior cavity with a gefitinib phosphate or gefitinib sulfate and an interior cavity aqueous medium is provided. In some embodiments, the liposome includes an interior cavity aqueous medium with an interior cavity pH of about 6 to 8. In other embodiments, the interior cavity pH is about 7 to 8. In other embodiments, the interior cavity pH is about 7.4 to 7.6. In some embodiments, the gefitinib phosphate or gefitinib sulfate is present at a therapeutically effective amount.

In one aspect, a liposome including an interior cavity with a LY 29004 phosphate or LY 29004 sulfate and an interior cavity aqueous medium is provided. In some embodiments, the liposome includes an interior cavity aqueous medium with an interior cavity pH of about 6 to 8. In other embodiments, the interior cavity pH is about 7 to 8. In other embodiments, the interior cavity pH is about 7.4 to 7.6. In some embodiments, the LY 29004 phosphate or LY 29004 sulfate is present at a therapeutically effective amount.

In one aspect, a liposome including an interior cavity with a pitavastatin phosphate or pitavastatin sulfate and an interior cavity aqueous medium is provided. In some embodiments, the liposome includes an interior cavity aqueous medium with an interior cavity pH of about 6 to 8. In other embodiments, the interior cavity pH is about 7 to 8. In other embodiments, the interior cavity pH is about 7.4 to 7.6. In some embodiments, the pitavastatin phosphate or pitavastatin sulfate is present at a therapeutically effective amount.

In one aspect, a liposome including an interior cavity with a TG101209 phosphate or TG101209 sulfate and an interior cavity aqueous medium is provided. In some embodiments, the liposome includes an interior cavity aqueous medium with an interior cavity pH of about 6 to 8. In other embodiments, the interior cavity pH is about 7 to 8. In other embodiments, the interior cavity pH is about 7.4 to 7.6. In some embodiments, the TG101209 phosphate or TG101209 sulfate is present at a therapeutically effective amount.

### V. PHARMACEUTICAL FORMULATIONS

The methods and compositions provided herein are useful in perparing pharmaceutical liposome compostions. In one aspect, a pharmaceutical composition prepared according to the methods provided herein including embodiments thereof is provided. In some embodiments, the present invention can include a liposome composition and a physiologically (i.e., pharmaceutically) acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized liposome compositions.

The liposome composition of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular, intravenous, intramuscular, intratumoral, intradermal, intraperitoneal, and subcutaneous routes, can be prepared. Such formulations can include aqueous and non-aqueous, isotonic sterile injection solutions, which can contain antioxidants, buffers, bacteriostats, and solutes that render the formulation isotonic with the blood of the intended recipient. Formulations for injection can also include aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration and intravenous administration are the preferred methods of administration. The formulations of liposome compositions can be presented in unit-dose or multi-dose sealed containers, such as ampoules and vials.

Suitable formulations for rectal administration include, for example, suppositories, which includes an effective amount of a packaged liposome composition with a suppository base. Suitable suppository bases include natural or synthetic triglycerides or paraffin hydrocarbons. In addition, it is also possible to use gelatin rectal capsules which contain a combination of the liposome composition of choice with a base, including, for example, liquid triglycerides, polyethylene glycols, and paraffin hydrocarbons.

The pharmaceutical preparation is preferably in unit dosage form. In such form the preparation is subdivided into unit doses containing appropriate quantities of the active component, e.g., a liposome composition. The unit dosage form can be a packaged preparation, the package containing discrete quantities of preparation. The composition can, if desired, also contain other compatible therapeutic agents.

### VI. METHODS OF TREATMENT

The methods and compositions provided herein are particularly useful in different diseases (e.g. cancer, inflammatory diseases, cholesterol diseases, neurological diseases, cardiovascular diseases). In one aspect, a method of treating a disease in a subject in need thereof is provided. The method includes administering to the subject a therapeutically effective amount of a pharmaceutical composition prepared according to the methods provided herein including embodiments thereof. In some embodiments, the disease is cancer. In other embodiments, the cancer is brain cancer. In some embodiments the disease is a cholesterol disease. In some embodiments the disease is an inflammatory disease. In some embodiments the disease is a neurological disease. In some embodiments the disease is a cardiovascular disease.

As used herein, the term “cancer” refers to all types of cancer, neoplasm, or malignant tumors found in mammals, including leukemia, carcinomas and sarcomas. Exemplary cancers include cancer of the brain (e.g. glioma, astrocytoma), breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The liposomes of the present invention may be administered to a subject in need thereof in a way that they may amass at a given site (target site) in a subject after administration, having ceased to systemically circulate within the subject. As used herein, the term “target site” refers to a location at which liposome accumulation and delivery of an active pharmaceutical ingredient is desired. In some cases, the target site can be a particular tissue or cell and may be associated with a particular disease state. In some cases, the accumulation may be due to binding of a specific biomarker at the target site by a liposome comprising a ligand that recognizes the biomarker. In some cases, the liposome accumulation may be due to the enhanced permeability and retention characteristics of certain tissues such as cancer tissues. Liposome accumulation may be assessed by any suitable means, such as compartmental analysis of test subjects or non-invasive techniques such as positron emission tomography or nuclear magnetic resonance imaging. However, one of skill in the art can plan the timing of liposome administration to a particular subject so as to allow for sufficient accumulation at a target site without directly measuring accumulation in the subject.

In therapeutic use for the treatment of cancer, the liposome compositions of the present invention including an effective amount of an active pharmaceutical ingredient can be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the liposome composition being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular liposome composition in a particular patient. Determination of the proper dosage for a particular situation is within the skill of the practitioner. Generally, treatment is initiated with smaller dosages which are less than the optimum dose of the liposome composition. Thereafter, the dosage is increased by small increments until the optimum effect under circumstances is reached. For convenience, the total daily dosage may be divided and administered in portions during the day, if desired.

### VII. EXAMPLES

### Animal Subjects

Athymic nu/nu mice of either sex, 5-6 weeks of age were used and 5 mice were housed in sterilized cages supplied with purified air passed through activated charcoal and HEPA filters. The mice were provided with autoclaved bedding, food, and water. All animal procedures were conducted in strict accordance with all appropriate regulatory standards under UCSD animal use protocol #S10005 which was reviewed and approved by the UCSD Institutional Animal Care and Use Committee (IACUC).

Cell Culture and Reagents:

A172, U87, U118 and U251 glioblastoma lines were maintained under standard culture conditions in Dulbecco's modified Eagle's medium (DMEM) supplemented with 10% FBS, GBM4 and 8 human tumorspheres were cultured in human basal neural stem cell medium supplemented with EGF, FGF and supplement medium. Dioleoylphosphatidylethanolamine (DOPE), cholesterol, distearoylphosphatidylcholine (DSPC) and mPEG2000 were purchased from Avanti Polar Lipids. Staurosporine was purchased from LC laboratories. Sephadex G50 from GE health care, Sepharose CL6B, DMEM were purchased from Sigma.

Preparation of Liposomes:

The liposome formulation included cholesterol, DOPE, DSPC, DSPE-mPEG2000 (6:6:6:1 molar ratio) in chloroform, and was evaporated under argon gas (Murphy, E. A. et al., Mol Cancer Ther 2011, 10 (6), 972-82). The dried lipid film was hydrated with a buffer containing either ammonium phosphate, ammonium sulfate, sodium phosphate, or sodium sulfate (300 mM, pH 7.4) and vortexed for 1 minute to remove any adhering lipid film. Sonication in a bath sonicator (ULTRAsonik 28X) for 1 minute at room temperature produced multilamellar vesicles (MLV). MLVs were then sonicated with a Ti-probe (Branson 450 sonifier) for 2 minutes to produce small unilamellar vesicles (SUV), indicated by the formation of a translucent solution. Extrusion through a 100 nm pore size polycarbonate filter (Whatman) was the final stage of a stepwise series of extrusions to reduce SUV size.

Reverse pH Gradient for Drug Encapsulation:

The prepared liposomes with internal buffer at pH 3 or 7.4 were passed through a Sephadex G-50 column equilibrated with HEPES buffered saline (HBS; 20 mM HEPES, 150 mM NaCl, pH 7.4) to exchange the external buffer. Staurosporine in 10% citric acid was added to the liposome suspension, and the external buffer pH was changed to either 3, 5, or 7.4. The solution was heated at 50° C. for 20 minutes, left to stand 4 hours at room temperature, and then applied to a Sephadex G-50 column and eluted with MilliQ water. Encapsulated staurosporine was quantified by measuring absorbance of the liposome suspension at 294 nm on a spectrophotometer, and interpolating concentration from a standard curve of free staurosporine.

Liposome Physical Characterization: The liposome suspension was diluted in 1/10 in MilliQ water and a 100 μl aliquot was sized using light backscattering (Malvern Zetasizer, ZEN 3600). The same instrument measured particle net charge expressed in mV. Size and surface ζ-potential were obtained from three repeat measurements with a backscattering angle of 173°. Liposome morphology and size were further characterized using scanning electron microscopy (SEM). Samples were prepared by applying 5 μl droplets of the liposome suspension onto a polished silicon wafer. After drying the droplets at room temperature overnight, the wafer was coated with chromium, and then imaged on a Philips XL-30 electron microscope to 30,000X.

In vitro Drug release studies: Staurosporine loaded liposomes were incubated in PBS or human serum at room temperature. The staurosporine concentration remaining in the liposomes was at 0.5, 1, 2, 4, 6, 12, 24, 48, and 72 hours. Separation of the liposomes from both PBS and serum was performed by size exclusion chromatography (SEC). The PBS liposome suspension was applied to a Sephadex G-50 column, while a sepharose CL6B column was used for serum, both eluted with PBS (Yamauchi, M. et al., Int J Pharm 2008, 351 (1-2), 250-8). After separation the concentration of staurosporine within liposomes incubated in PBS was determined spectrophotometrically at 294 nm. The liposomes separated from serum were lyophilized, dissolved in HPLC grade methanol, centrifuged, and the supernatant collected following by evaporation. The evaporation residue was dissolved in 100% HPLC grade acetonitrile, and HPLC (Agilent HPLC) performed using a 70% acetonitrile-water mobile phase to measure staurosporine.

Activity of Encapsulated Staurosporine Against Glioblastoma Cells In Vitro:

Applicants evaluated the cytotoxic effect of staurosporine encapsulating liposomes and free staurosporine on a range of established human glioblastoma cell lines including A172, U251, U118, and U87 cells. All cells were grown in 96 well plates in complete medium with 10% FBS at 37° C., and then either free staurosporine or staurosporine liposomes were added and the cells incubated for another 72 hours. Applicants also established human glioblastoma cancer stem cells from fresh surgical isolates using stem cell media methods as previously reported and tested staurosporine cytotoxicity with these lines. For the experiments with free staurosporine, 10 mmol/L stocks were first serially diluted in DMSO then with medium, to avoid precipitation. Cell viability was quantified using the MTT assay (Rajesh, M. et al., J Am Chem Soc 2007, 129 (37), 11408-20). Briefly, the absorbance at 540 nm was measured after adding MTT (Sigma-Aldrich). Results were expressed as percent viability=[A540(treated cells)−background/A540(untreated cells)−background]×100%. Dose-response curves were plotted by using GraphPad® Prism software and EC50 values were calculated.

In Vivo Activity of Encapsulated Staurosporine Measured with Flank Tumor Model:

Athymic nu/nu mice 5-6 weeks old were subcutaneously inoculated in the right and left flanks with 4 million U87 cells, and the resultant tumors were allowed to grow to 40-50 mm³. The mice were sorted so that the treatment and control groups had the same average tumor size. Mice were injected intravenously with liposomally encapsulated staurosporine, or with PBS; it should be noted that free staurosporine is cleared immediately, and the doses required to bypass this effect are acutely toxic. The encapsulated staurosporine groups received a 0.8 mg/kg staurosporine dose 3 times per week for 3 weeks. The tumors were measured weekly using calipers and volume was calculated using the standard formula, V=(Length×[Width])/2. The mice were sacrificed when the tumors attained 1500 mm³. Body weights were measured before and after 3 weeks of treatment.

Recurrent Tumor Study with Encapsulated Staurosporine:

To confirm that suppression of tumor growth was due to treatment with liposomal staurosporine and not an artifact from failure of the tumors to thrive, the treatment was stopped at 3 weeks in the encapsulated staurosporine group, and the tumors allowed to regrow over a 5 week period. When the tumors reached 120 mm³, treatment was resumed for one week during which three IV doses of either PBS, or encapsulated staurosporine at 0.8 mg/Kg, were given. The tumors were measured weekly for the next two weeks, and then the mice were sacrificed in accordance with Applicants' IACUC protocol. Body weights were taken before and after this second treatment course.

### Results

Liposome Physical Characterization:

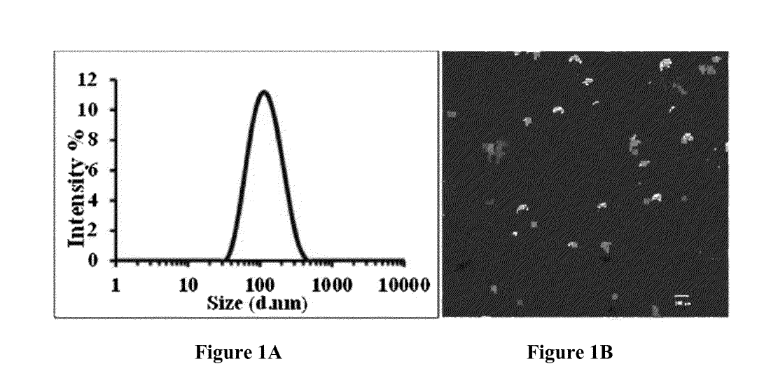

The average liposomal size was 108±1.1 nm (three measurements), the zeta potential or net charge was close to the desired neutrality at 2.18±2.4 (ten measurements). SEM data confirmed that the staurosporine loaded liposomes were spherical, intact and averaged approximately 108 nm in diameter (FIGS. 1a and 1b).

Encapsulation Efficiency:

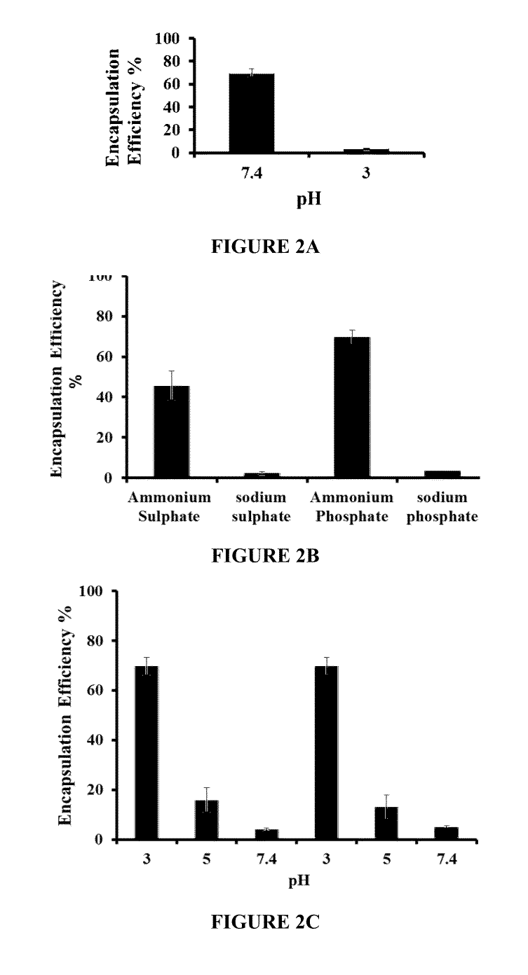

Reverse pH gradient with ammonium based buffers produced effective encapsulation: FIG. 2a shows the effect of internal pH and 2b shows the effects of internal buffer composition. Consistent with previous reports, Applicants obtained poor loading of staurosporine (≈5%) with an internal pH of 3 and external pH of 7.4 (FIG. 2c). Internal pH levels of 5 with an external pH of 7.4 also resulted in very low encapsulation rates. The best encapsulation efficiencies were achieved when the internal buffer pH was 7.4 and was based on ammonium phosphate, or ammonium sulfate, with rates of 70% and 65%, respectively, while sodium phosphate and sodium sulfate produced very low encapsulation of 3-4% (FIGS. 2a, 2b, and 2c). Externally, HEPES and PBS buffers were associated with the same encapsulation efficiency (FIG. 2c).

Optimal Drug to Lipid Ratio for Efficient Encapsulation:

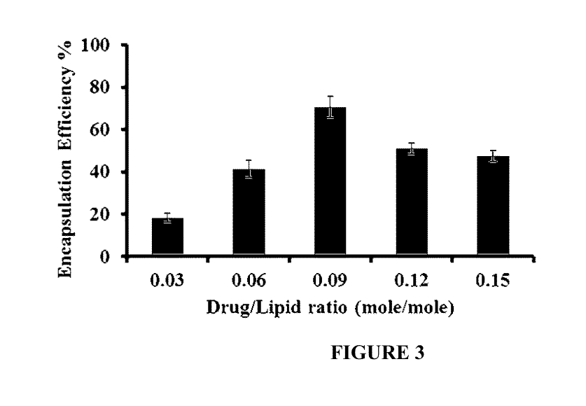

In order to determine how escalating drug concentrations would affect loading efficiency and/or formulation stability, staurosporine was added in drug-to-lipid ratios of 0.03, 0.06, 0.09, 0.12 or 0.15 (mole/mole). Staurosporine uptake into the liposomes was measured as described in the methods, and as shown in FIG. 3 an optimal drug to lipid ratio was obtained. Liposomal loading capacity was highest when drug to lipid ratio was 0.09 (mole/mole), with a peak value of 70% which is higher than for previously described formulations (Yamauchi, M. et al., Int J Pharm 2008, 351 (1-2), 250-8).

In Vitro Drug Release:

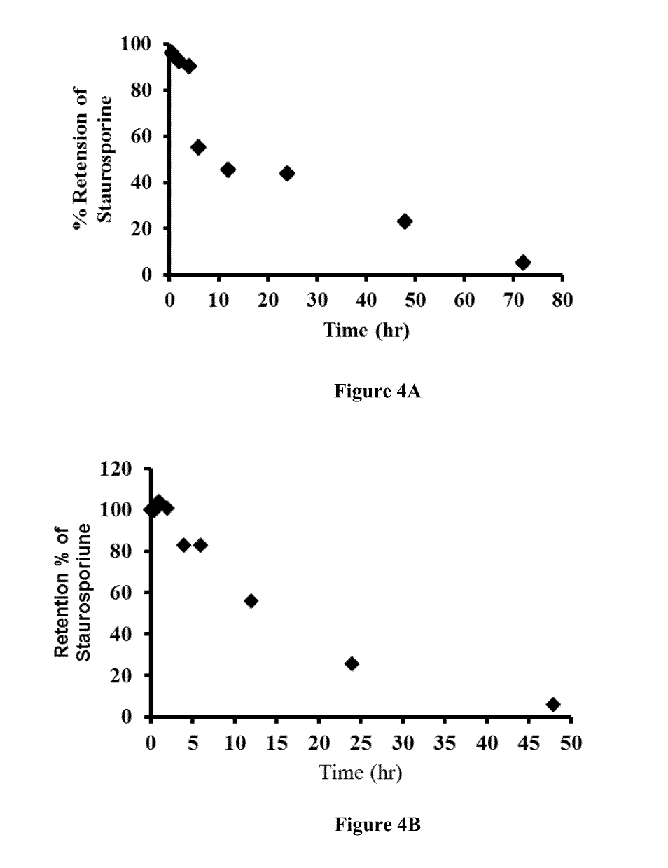

In vitro staurosporine release studies revealed that the encapsulating liposomes were stable for several hours with comparatively little leakage of payload. FIGS. 4a and 4b reveal that after 3 hours of incubation in PBS or human serum, the liposomes retained almost 100% of the initial staurosporine payload. By 4-6 hours of incubation in serum they retained 80% of the staurosporine, and after 12 hours 60% of the compound was still contained. Applicants expect that after 8 hours the PEGylated liposomes will begin to be cleared. The key point is that since liposomal delivery of payload to tumors is efficient due to the enhanced permeability and retention (EPR) effect and also potentially enhanced by targeting, only a low, subtoxic total dose of staurosporine needs to be administered, and thus some leakage of this staurosporine from the liposomes over 24 hours is not expected to be toxic.

Activity of Encapsulated Staurosporine Against Glioblastoma Cell Lines In Vitro:

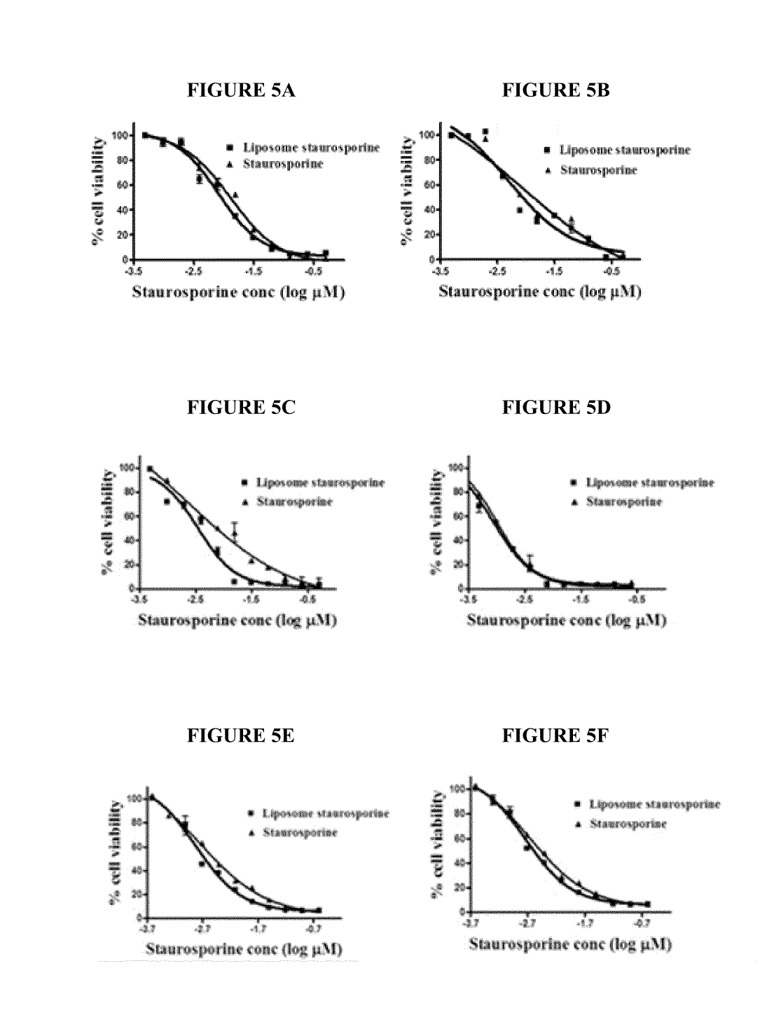

Encapsulation of staurosporine within liposomes did not impede its cytotoxic effect. Applicants evaluated the inhibitory effect of staurosporine encapsulating liposomes on range of established and freshly derived human glioblastoma cell lines (FIGS. 5a-f). The results depicted in FIGS. 5a-f show that the EC50 of both staurosporine contained in liposomes and free staurosporine was comparable and potent in all cell lines, and ranged from 1-10 nM. This means that encapsulated staurosporine was taken up and released within the tumor cells.

Encapsulated Staurosporine Exhibits In Vivo Anti-Tumor Activity with No Overt Evidence of Toxicity:

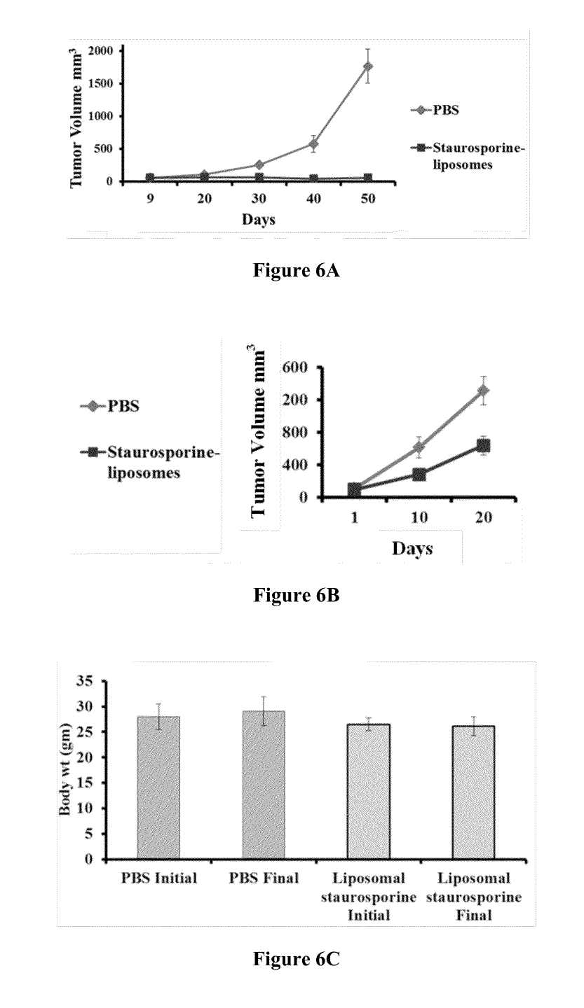

Applicants then addressed the essential question of whether a subtoxic dose of staurosporine encapsulated in liposomes could remain in the blood circulation long enough to be delivered to a tumor in vivo causing a significant anti-tumor effect. If the 0.8 mg/Kg dose Applicants tested were to be injected as free staurosporine, it would very likely be 99% cleared after only one circulatory pass, and after subsequent passes a negligible amount would be present (Gurley L R et al., Staurosporine analysis and its pharmacokinetics in the blood of rats; Los Alamos National Laboratory: Los Alamos, July 1994, 1994). A much higher dose of free staurosporine, 5 mg/Kg, was acutely lethal. Applicants demonstrated that 0.8 mg/kg of staurosporine delivered by non-targeted liposomes completely suppressed tumor growth in the flank model for 50 days after the tumors were already well established (FIG. 6a). Moreover as shown in FIG. 6b the mice treated with 0.8 mg/kg of encapsulated staurosporine or PBS had no statistically significant weight change and exhibited no obvious deleterious effects. In contrast PBS treated mice exhibited rapid growth of their flank tumors. Future, more comprehensive studies will use even lower doses of staurosporine alone, and in combination with other agents.

When treatment was stopped and the staurosporine treated mice experienced tumor regrowth during the ensuing 5 weeks, they were separated into two groups and treated with either PBS or liposomal staurosporine for one week, and then the animals were only observed for the next two weeks. It can be seen from FIG. 6c that three treatments with liposomal staurosporine significantly slowed the growth of the tumors, which had become very large, relative to PBS treatment.

The objective of this study was to efficiently load staurosporine into liposomes so that a low dose could be systemically administered while at the same time selectively concentrated at a tumor to attain a therapeutic local dose. Staurosporine is a potent anti-tumor agent but at relatively non-toxic systemic dose levels it is almost entirely removed from the circulation in one complete circuit of the blood, while higher doses have significant toxicity due to the non-selectivity of this pan kinase inhibitor (Gurley L R et al., Staurosporine analysis and its pharmacokinetics in the blood of rats; Los Alamos National Laboratory: Los Alamos, July 1994, 1994). These obstacles can potentially be overcome by loading staurosporine into liposomes to (1) shield it from protein binding and prevent clearance, and (2) selectively deliver it to tumors via leaky tumor vasculature, a well-documented process termed the enhanced permeability and retention (EPR) effect (Wang, A. Z. et al., Nanoparticle Delivery of Cancer Drugs. Annu Rev Med 2011).

Liposomal loading based on chemical gradients to drive the accumulation of a drug payload represents a key advance in terms of efficiency and simplicity (Madden, T. D. et al.; Chem Phys Lipids 1990, 53 (1), 37-46; Stensrud, G. et al.; Int J Pharm 2000, 198 (2), 213-28). This gradient based approach was considerably advanced by the concept of using ammonium ion liposomal transmembrane gradients to form a self-sustaining pH gradient (Haran, G. et al., Biochim Biophys Acta 1993, 1151 (2), 201-15). Attempted gradient based loading of liposomes with staurosporine has yielded poor results, but gradient methods are quite desirable in terms of overall efficiency, so in the present study Applicants focused on the strategy of adapting gradient methodology (Yamauchi, M. et al., Biol Pharm Bull 2005, 28 (7), 1259-64). Applicants initially examined the role of external versus internal pH and found that that a reversal of the typically used pH gradient dramatically increased staurosporine loading efficiency. An internal versus external buffer pH of 7.4 and 3, respectively, was effective at driving staurosporine into liposomes. Applicants next explored the effects of various buffers, and the drug to lipid ratio. HEPES and PBS external buffers had equal effect, while internal buffers based on ammonium salts were associated with much higher staurosporine loading than were sodium salts, with the best results acquired using ammonium phosphate. Furthermore, Applicants found an optimal drug (staurosporine) to lipid ratio in terms of the efficiency of liposomal staurosporine loading.

Applicants' reverse loading methodology also offered an advantage in terms of liposomal staurosporine retention. Prevention of premature release of drug payload by circulating liposomes is an essential requirement, but payload release still has to occur at the tumor site. Previous gradient loading methods only supported the liposomal retention of weakly basic anthracyclines such as doxorubicin and to some extent the campothecin analogues (Madden, T. D. et al.; Chem Phys Lipids 1990, 53 (1), 37-46; Stensrud, G. et al.; Int J Pharm 2000, 198 (2), 213-28). Other drug chemotypes such as cationic compounds could not be retained. The reverse pH gradient loading methodology Applicants describe resulted in stable liposomal staurosporine encapsulation with essentially all compound still contained after 3 hours of incubation in human serum. As a result the liposomal outer shell did not have to be modified by extensive cross-linking and the addition of stabilizers to slow payload efflux. Despite stable liposomal retention the release of staurosporine at the tumor site appeared to take place, as evidenced by the significant anti-tumor effect Applicants observed in the glioblastoma flank tumor model.

An important demonstration in this study was that staurosporine could be efficiently loaded into liposomes. Furthermore, Applicants obtained evidence of antitumor activity at a low, apparently non-toxic systemic dose, showing that the compound was not cleared before tumor accumulation and a significant anti-tumor effect developed. The present report is intended to provide the basis for later, more comprehensive studies on the efficacy and toxicology of low dose encapsulated staurosporine as a potential glioblastoma therapeutic. In addition, the use of reverse gradient loading for liposomal encapsulation may be applicable to other drug chemotypes.

### VIII. EMBODIMENTS

### Embodiment 1

A method of forming a liposomally encapsulated drug, said method comprising: (i) contacting an unloaded liposome with a drug in an exterior aqueous medium at an exterior aqueous medium pH, wherein said unloaded liposome comprises an interior cavity aqueous medium with an interior cavity pH at least 2 units higher than the exterior aqueous medium pH; and (ii) allowing said drug to move from said exterior aqueous medium to said interior cavity thereby forming a liposomally encapsulated drug.

### Embodiment 2

The method of embodiment 1, wherein said interior cavity pH is from about 5 to about 9.

### Embodiment 3

The method of embodiment 1, wherein said interior cavity pH is from about 6 to about 8.

### Embodiment 4

The method of embodiment 1, wherein said interior cavity pH is from about 7 to about 8.

### Embodiment 5

The method of embodiment 1, wherein said interior cavity pH is from about 7.4 to about 7.6.

### Embodiment 6

The method of one of embodiments 1 to 5, wherein said exterior aqueous medium pH is about 1 to 4.

### Embodiment 7

The method of one of embodiments 1 to 5, wherein said exterior aqueous medium pH is about 2 to 4.

### Embodiment 8

The method of one of embodiments 1 to 5, wherein said exterior aqueous medium pH is about 2.5 to 3.5.

### Embodiment 9

The method of one of embodiments 1 to 5, wherein said exterior aqueous medium pH is about 3.

### Embodiment 10

The method of one of embodiments 1 to 9, wherein the drug has a pKa at least 2 units higher than the exterior aqueous medium pH.

### Embodiment 11

The method of one of embodiments 1 to 10, wherein the drug is present in the exterior aqueous medium as an exterior aqueous medium drug salt and is present in the interior cavity as an interior cavity drug salt.

### Embodiment 12

The method of one of embodiments 1 to 11, wherein the drug is staurosporine.

### Embodiment 13

The method of embodiment 12, wherein the aqueous medium drug salt is staurosporine citrate and said interior cavity drug salt is staurosporine phosphate or staurosporine sulfate.

### Embodiment 14

The method of one of embodiments 1 to 11, wherein the drug is doxorubicin.

### Embodiment 15

The method of embodiment 14, wherein the aqueous medium drug salt is doxorubicin citrate and said interior cavity drug salt is doxorubicin phosphate or doxorubicin sulfate.

### Embodiment 16

The method of one of embodiments 1 to 11, wherein the drug is dasatinib.

### Embodiment 17

The method of embodiment 16, wherein the aqueous medium drug salt is dasatinib citrate and said interior cavity drug salt is dasatinib phosphate or dasatinib sulfate.

### Embodiment 18

The method of one of embodiments 1 to 11, wherein the drug is imatinib.

### Embodiment 19

The method of embodiment 18, wherein the aqueous medium drug salt is imatinib citrate and said interior cavity drug salt is imatinib phosphate or imatinib sulfate.

### Embodiment 20

The method of one of embodiments 1 to 11, wherein the drug is gefitinib.

### Embodiment 21

The method of embodiment 20, wherein the aqueous medium drug salt is gefitinib citrate and said interior cavity drug salt is gefitinib phosphate or gefitinib sulfate.

### Embodiment 22

The method of one of embodiments 1 to 11, wherein the drug is a statin.

### Embodiment 23

The method of embodiment 22, wherein the drug is pitavastatin.

### Embodiment 24

The method of embodiment 23, wherein the aqueous medium drug salt is pitavastatin citrate and said interior cavity drug salt is pitavastatin phosphate or pitavastatin sulfate.

### Embodiment 25

The method of one of embodiments 1 to 11, wherein the drug is a P13 kinase inhibitor.

### Embodiment 26

The method of embodiment 25, wherein the P13 kinase inhibitor is LY-29004.

### Embodiment 27

The method of embodiment 26, wherein the aqueous medium drug salt is LY-29004 citrate and said interior cavity drug salt is LY-29004 phosphate or LY-29004 sulfate.

### Embodiment 28

The method of embodiment 11, wherein said exterior aqueous medium drug salt is more soluble in said exterior aqueous medium than said interior cavity drug salt in said interior cavity aqueous medium.

### Embodiment 29

The method of embodiment 28, wherein said interior cavity drug salt is in crystalline form and said exterior aqueous medium drug salt is solubilized in said exterior aqueous medium.

### Embodiment 30

The method of embodiment 1, wherein said allowing is performed at about 50° C.

### Embodiment 31

The method of embodiment 30, wherein said allowing is performed for about 20 minutes to about 60 minutes.

### Embodiment 32

The method of embodiment 30, wherein said allowing is performed for about 30 minutes to about 60 minutes.

### Embodiment 33

The method of embodiment 30, wherein said allowing is performed for about 40 minutes to about 60 minutes.

### Embodiment 34

The method of embodiment 30, wherein said allowing is performed for about 50 minutes to about 60 minutes.

### Embodiment 35

The method of embodiment 1, wherein said unloaded liposome and said drug are present at a ratio of about 0.09 mol of liposome/mol of drug.

### Embodiment 36

A liposome comprising an interior cavity with a staurosporine phosphate or staurosporine sulfate and an interior cavity aqueous medium.

### Embodiment 37

The liposome of embodiment 36, comprising an interior cavity aqueous medium with an interior cavity pH of about 6 to 8.

### Embodiment 38

The liposome of embodiment 37, wherein the interior cavity pH is about 7 to 8.

### Embodiment 39

The liposome of embodiment 38, wherein the interior cavity pH is about 7.4 to 7.6.

### Embodiment 40

The liposome of embodiment 36, wherein said staurosporine phosphate or staurosporine sulfate is present at a therapeutically effective amount.

### Embodiment 41

A pharmaceutical composition prepared according to the method of any one of embodiments 1 to 35.

### Embodiment 42

A method of treating a disease in a subject in need thereof, said method comprising, administering to said subject a therapeutically effective amount of a pharmaceutical composition prepared according to the method of any one of embodiments 1 to 35.

### Embodiment 43

The method of embodiment 42, wherein said disease is cancer.

### Embodiment 44

The method of embodiment 43, wherein said cancer is brain cancer.

### Embodiment 45

The method of embodiment 42, wherein said disease is a cholesterol disease.

1. A method of forming a liposomally encapsulated drug, said method comprising:
(i) contacting an unloaded liposome with a drug in an exterior aqueous medium at an exterior aqueous medium pH, wherein said unloaded liposome comprises an interior cavity aqueous medium with an interior cavity pH at least 2 units higher than the exterior aqueous medium pH; and
(ii) allowing said drug to move from said exterior aqueous medium to said interior cavity thereby forming a liposomally encapsulated drug.

2. (canceled)

3. The method of claim 1, wherein said interior cavity pH is from about 6 to about 8.

4. (canceled)

5. (canceled)

6. The method of claim 1, wherein said exterior aqueous medium pH is about 1 to 4.

7. (canceled)

8. (canceled)

9. (canceled)

10. The method of claim 1, wherein the drug has a pKa at least 2 units higher than the exterior aqueous medium pH.

11. The method of claim 1, wherein the drug is present in the exterior aqueous medium as an exterior aqueous medium drug salt and is present in the interior cavity as an interior cavity drug salt.

12. The method of claim 11, wherein the drug is selected from the group consisting of staurosporine, doxorubicin, dasatinib, imatinib, gefitinib, statin, and a P13 kinase inhibitor.

13. The method of claim 12, wherein the aqueous medium drug salt is staurosporine citrate and said interior cavity drug salt is staurosporine phosphate or staurosporine sulfate.

14-27. (canceled)

28. The method of claim 11, wherein said exterior aqueous medium drug salt is more soluble in said exterior aqueous medium than said interior cavity drug salt in said interior cavity aqueous medium.

29. (canceled)

30. The method of claim 1, wherein said allowing is performed at about 50° C.

31-35. (canceled)

36. A liposome comprising an interior cavity with a staurosporine phosphate or staurosporine sulfate and an interior cavity aqueous medium.

37. The liposome of claim 36, comprising an interior cavity aqueous medium with an interior cavity pH of about 6 to 8.

38. (canceled)

39. (canceled)

40. The liposome of claim 36, wherein said staurosporine phosphate or staurosporine sulfate is present at a therapeutically effective amount.

41. A pharmaceutical composition prepared according to the method of claim 1.

42. A method of treating a disease in a subject in need thereof, said method comprising, administering to said subject a therapeutically effective amount of a pharmaceutical composition prepared according to the method of claim 1.

43. (canceled)

44. The method of claim 42, wherein said disease is brain cancer.

45. (canceled)
